(19) United States
(12) Patent Application Publication
(10) Pub. No.: US 2023/0301885 A1
(43) Pub. Date: Sep. 28, 2023

(54) NOVEL USE

(71) Applicant: DSM IP ASSETS B.V., Heerlen (NL)

(72) Inventor: Sebastien MONGIAT, Kaiseraugst (CH)

(21) Appl. No.: 18/009,603

(22) PCT Filed: Jun. 10, 2021
(86) PCT No.: PCT/EP2021/065581

§ 371 Date: Dec. 9, 2022

(30) Foreign Application Priority Data
Jun. 12, 2020 (EP) 20179759.4

(51) Int. Cl. A61K 8/34; A61Q 19/00; A61Q 17/00

(52) U.S. Cl. CPC A61K 8/345; A61Q 17/005; A61Q 19/007; A61K 2800/70

(57) ABSTRACT

The present invention relates to the use of 1,3-propanediol as an agent to balance the growth of commensal skin microorganisms of dry and sebaceous skin and thus to foster the maintenance of a healthy skin barrier.

The present invention relates to the use of 1,3-propanediol as an agent to balance the growth of commensal skin microorganisms of dry and sebaceous skin and thus to foster the maintenance of a healthy skin barrier.

One of the main functions of the skin is to form a physical barrier (commonly called the skin barrier) that, in addition to its protective role with respect to the environment by preventing the penetration of aggressive microbial or chemical elements, must provide the maintaining of the physiological medium of the organism by limiting water loss, thanks to a relative hydrophobicity.

It is well known that the surface of the skin is colonized by a great variety of microorganisms which form the skin microbiome (often also called skin microbiota). Resident microbiota are found in the upper parts of the epidermidis and congregated in and around the hair follicle. The majority of the microorganisms on human skin are commensal or mutualistic bacteria. However, the microbiota may also include or be exposed to pathogenic bacteria such as S. aureus. Commensals generally live in peaceful coexistence with the host while benefiting from the sheltered ecological niche. Mutualistic microbes offer benefits to their host. Pathogenic microbes may be harmful to their host, in particular if their presence exceeds certain thresholds.

It is by now well established that the skin microbiome, i.e. the surface commensal microbial community has an impact on the skin barrier. Thus, a disbalance in the natural microbiome may lead to a weakening or even break down of the skin barrier.

Each human skin area is colonized by a particular microbial composition. For keeping the skin healthy the maintenance of this biodiversity is particularly important.

As known to a person skilled in the art, the face area is characterized by a more sebaceous skin where main representative bacterial and fungal strains are Cutibacterium acnes, Staphylococcus epidermidis, Staphylococcus hominis, Staphylococcus capitis, Streptococcus mitis, Corynebacterium simulans and Malassezia globosa. On the other side, the body area is characterized by a more dry skin, compared to face or even scalp, where main representative bacterial and fungal strains are Corynebacterium tuberculostearicum, Cutibacterium acnes, Streptococcus mitis, Streptococcus oralis, Micrococcus luteus and Malassezia globosa.

Accordingly it is well accepted nowadays that the microbiota of sebaceous skin is mainly composed of Cutibacterium acnes (C. acnes), Staphylococcus epidermidis (S. epidermidis), Staphylococcus hominis (S. hominis), Staphylococcus capitis (S. capitis), Streptococcus mitis (S. mitis), Corynebacterium simulans (C. simulans ) and Malassezia globosa (M. globosa). and the microbiota of dry skin is mainly composed of Corynebacterium tuberculostearicum (C. tuberculostearicum), Cutibacterium acnes (C. acnes), Streptococcus mitis (S. mitis), Streptococcus oralis (S. oralis), Miccrococcus luteus (M. luteus) and Malassezia globosa (M. globosa).

M. globosa is a fungi whereas C. tuberculostearicum, M. luteus, C. acnes (formerly Propionibacterium acnes), S. mitis, S. oralis, S. hominis, S. capitis, C. simulans and S. epidermidis are Gram positive bacteria which are all considered as commensal (skin) microbes.

Staphylococcus aureus (S. aureus) on the other hand is a Gram positive bacterium which is a pathogenic microbe and which is redoubtable particularly due to the emergence of antibiotic-resistant strains of S. aureus such as methicillin-resistant S. aureus (MRSA) leading to a worldwide problem in clinical medicine. Despite much research and development, no vaccine for S. aureus has been approved.

1,3-Propanediol (also referred to herein as 1,3-PDO) is known to have an antimicrobial effect on the pathogenic microbes S. aureus and E. coli. However nothing is known about its effect on other microbes, let alone on the commensal microbes of dry or sebaceous skin.

CN106361684 discloses specific compositions for adjusting skin surface microbial balance, which compositions comprise at least oligomeric fructose (3-15 parts), monosaccharides (5-12 parts), lactobacillus/ soy (bean) milk filtrate (1-8 parts) and polyhydric alcohol(s) (30-50 parts), wherein the polyhydric alcohols (e.g. 1,2-hexanediol, propylene glycol and pentylene glycol) are described to have an inhibitory action for various stimulus.

The understanding of the interaction of cosmetic products with skin microbes, has gained more and more importance in the last few years. By now it has been well established that extrinsic factors, such as hygiene practices and/or environmental conditions can shift the natural equilibrium of the skin microbiota to a dysbiotic state, which in turn may lead to a weakening or even a breakdown of the skin barrier. Such compromised skin, however, allows for an increased penetration of exogenous substances, such as e.g. bacteria, polluting agents, irritant agents or allergenic substances, which in turn may cause mild to severe adverse health effects such as skin irritation, allergic reactions or even infections.

Accordingly, ingredients of a cosmetic product should not lead to a disbalance of the skin microbiome neither by significantly modifying the natural distribution of the commensal microbes amongst each other nor by (over)stimulating the growth of pathogenic microbes.

Thus, there is a need of ingredients which, next to fulfilling their envisaged role in the respective composition, furthermore foster the maintenance of a healthy skin microbiome, in particular in view of the specific microbiome of dry or sebaceous skin and thus support the maintenance of a healthy skin barrier and thus an overall healthy skin. Such ingredients, would preferably also reduce or even inhibit the growth of pathogenic bacteria, such as in particular S. aureus.

Therefore, the problem to be solved by the present invention is to offer an ingredient which has advantageous properties in the application to hair and skin, which, however, has also a good microbial growth balancing.

Surprisingly, it has now been found that 1,3-propanediol has a very good balancing effect specifically directed to the commensal microbes of dry or sebaceous skin as outlined above, while additionally providing a balancing effect of S. epidermidis over S. aureus, and is thus able to foster the maintenance of a healthy skin barrier in said skin types.

Thus, in a first aspect the present invention relates to use of 1,3-propanediol for balancing the microbial growth of commensal microbes on the skin, such as in particular microbes of dry or sebaceous skin, such as most preferably of M. globosa, C. tuberculostearicum, M. luteus, C. acnes, S. mitis, S. oralis, S. hominis, S. capitis, C. simulans and/or S. epidermidis, even more in particular over the growth of S. aureus.

In particular the balancing according to the present invention is
(1) a balancing of the growth of at least two microbes of dry skin or sebaceous skin by not unproportionally affecting the growth of anyone thereof, and/or
(2) a balancing of the growth of S. aureus in the presence of S. epidermidis by inhibiting the microbial growth of S. aureus to a higher extent than the one of S. epidermidis.

Advantageously, in all embodiments of the present invention the balancing of the growth of at least two microbes of dry or sebaceous skin by not unproportionally affecting the growth of anyone thereof takes place while at the same time the growth of S. aureus is inhibited to a higher extent than the one of S. epidermidis in the concomitant presence of both of said microbes, in particular when 1,3-propanediol is used in an amount selected in the range from 0.01 to 6.0 wt.-%, based on the total weight of the cosmetic composition.

Preferably in all embodiments of the present invention the balancing of the growth of microbes of dry skin or of sebaceous skin according to the present invention takes place between the dry skin microbes C. tuberculostearicum, C. acnes, S. mitis, S. oralis, M. luteus and M. globosa or the sebaceous skin microbes C. acnes, S. epidermidis, S. hominis, S. capitis, S. mitis, C. simulans M. globosa.

For sake of clarity, some terms as used in the present document are defined as follows:

The term ‘preparation’ or ‘formulation’ is used in this document as equivalent to the term ‘com position’.

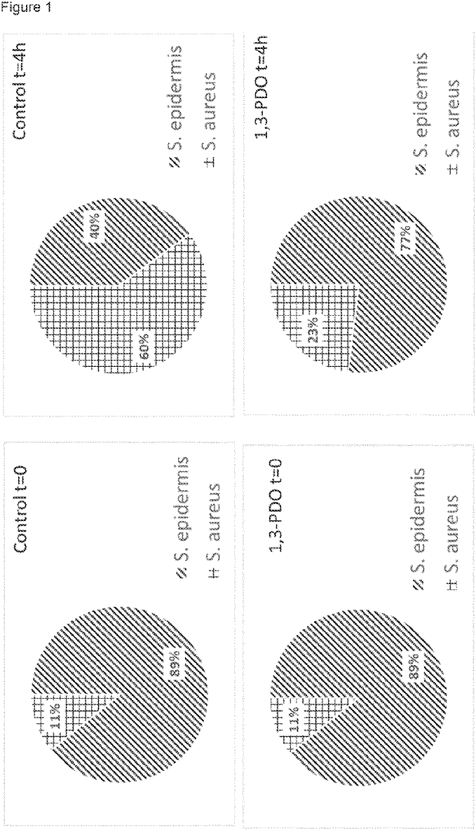

The term ‘share’ as used herein refers to the share of each (individual) microbe, based on the total number of microbes in the respective group of microbes in % (i.e. the at least two, preferably all dry or sebaceous skin microbes as defined herein, respectively S. epidermidis and ), either before or after inoculation (as indicated). The share is calculated using the colony count of the respective (individual) microbe divided by the sum of the colony counts of all microbes of the respective microbiota * 100%. Said shares can easily be represented by a pie diagram as illustrated in FIGS. 1 to 3, and is a good method of visualizing a distribution of individual microbes in a microbial co-culture.

Any ‘count(s)’ or ‘colony count(s)’ of microbe(s) is given in this document as colony-forming unit (cfu) per millilitre.

The term ‘skin’ as used in this document, is meant to include the external surface of mammals, especially humans and includes the skin and the scalp.

The term ‘control’ as used herein refers to the respective microbiota in the absence of 1,3-PDO.

The exogenous molecules can be in particular irritant substances (hygiene products, solvents, etc.) or allergenic substances (perfumes, house dust, microbial agents).

1,3 Propanediol has the formula

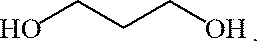

1,3 Propanediol is commercially readily available from different suppliers.

It is well understood, that the balancing according to the present invention always takes place in the mutual presence of the respective microbes, i.e. between the at least two, preferably all of the dry or sebaceous skin microbes as defined herein and/ or between S. epidermidis and S. aureus.

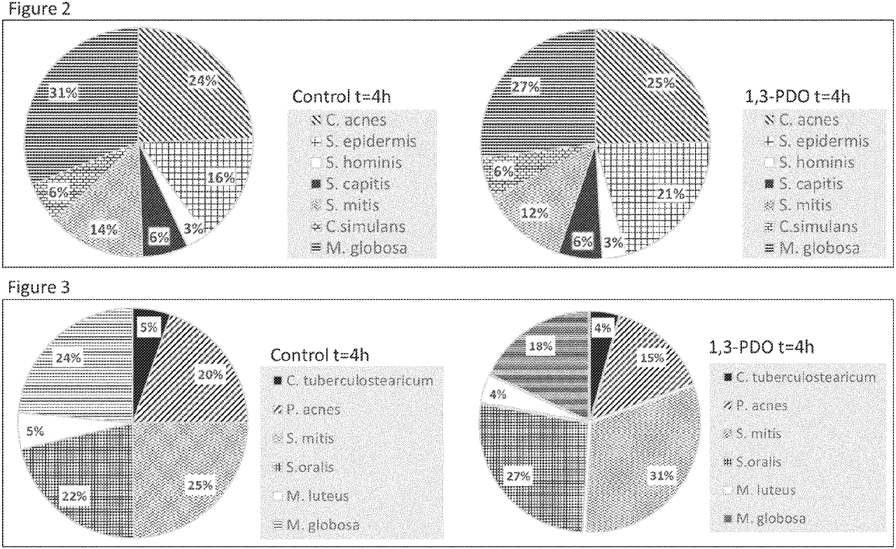

The term ‘by not unproportionally affecting the growth of anyone thereof’ refers to a balancing wherein the natural diversity of the respective skin microbiome is maintained compared to the control, as illustrated in FIGS. 2 and 3. Furthermore, in an even more preferred embodiment, the absolute (total) number of microbes is also not significantly altered by the ingredient.

Thus, in particular the term ‘by not unproportionally affecting the growth of anyone thereof’ refers to a balancing of at least two, preferably all dry skin or sebaceous skin microbes as defined herein in the mutual presence thereof, wherein only small changes in the difference in the (individual) shares of the microbes compared to the corresponding shares of the control are observed. Preferably, the small changes in the (individual) share (after 4 h of incubation at 37° C.) is less than ±15%, in particular less than ±10%, most in particular less than ±7.5%, compared to the control. Said change in the share is calculated as: share of the respective microbe within the group of at least two, preferably all microbes of dry or sebaceous skin in the presence of 1,3-PDO [%] minus share of the same microbe within the same group of microbes in the absence of 1,3-PDO [%], both after 4 h of incubation at 37° C. as illustrated in the example.

It is furthermore preferred that the total (i.e. the absolute) number of microbes in the respective microbiota (i.e. the sum of at the least two, preferably all microbes of dry or sebaceous skin as defined herein) is not significantly affected by the ingredient compared to the corresponding control. Preferably, the difference in total number of said at least two, preferably all microbes, compared to the respective total number in the absence of 1,3-propanediol (i.e. the control), after 4 h of incubation at 37° C., is within a range of ±50%, preferably ±40%, more preferably ±15%, most preferably ±10%.

Even more preferably, the difference in the total (absolute) number of each of the at least two microbes with respect to the total number of each of the microbes in the absence of 1,3-propanediol after incubation at 37 h for 4 h is
A) for the microbiota of sebaceous skin within the range of
   ±15%, preferably +5% to -15%, more preferably 0% to -10%, most preferably -2.5% to -7.5% for C. acnes,
   ±35%, preferably +5% to +35%, more preferably +15% to + 30%, most preferably +20% to +30% for S. epidermidis,
   ±15%, preferably +0% to +15%, more preferably +2.5% to + 10%, most preferably +2.5% to +7.5% for S. hominis,
   ±20%, preferably 0% to -20%, more preferably -7.5% to -15%, most preferably -10% to -15% for S. capitis,
   ±25%, preferably -5% to -25%, more preferably -10% to -23%, most preferably -15% to -20% for S. mitis,
   ±15%, preferably 0% to -15%, more preferably -2.5% to -10%, most preferably -5% to -10% for C. simulans, and/or
   ±25%, preferably -5% to -25%, more preferably -10% to -23%, most preferably -15% to -23% for M. globosa.
B) for the microbiota of dry skin within the range of
   ±20%, preferably +0% to 20%, more preferably +5% to +15%, most preferably +7.5% to +15% for C. tuberculostearicum,
   ±15%, preferably 0% to +15%, more preferably +2.5% to +10%, most preferably +5% to +10% for C. acnes,
   ±75%, preferably 0% to +75%, more preferably +25% to +75%, most preferably +50% to +75% for S. mitis and S. oralis,
   ±20%, preferably +0% to 20%, more preferably +5% to +15%, most preferably +7.5% to +15% for M. luteus, and/ or
   ±15%, preferably 0% to +15%, more preferably +2.5% to +10%, most preferably +5% to +10% for M. globosa.

It is noted that it is not unusual that other ingredients lead to significantly higher changes, in the share and/ or the total number of microbes in a certain microbiota or even result in the disappearance of one or more of the microbes as outlined in the comparative example.

The ‘not unproportionally affected growth’ can also be assessed by determining an overall impact value, by
(1) assessing the impact of the ingredient on the individual microbe (i) and assigning an individual impact value x′(i) to said microbe as outlined below:
$$x \left(i\right) = \left|\frac{c o l o n y c o u n t s a m p l e m i c r o b e \left(i\right)}{c o l o n y c o u n t c o n t r o l m i c r o b e \left(i\right)} - 1\right| \qquad \text{formula (I)}$$
If x(i) is
   a) ≤ 0.1 then an integer x′(i) of 1 is assigned (no impact)
   b) >0.1 ≤ 0.5then an integer x′(i) of 2 is assigned (slight impact)
   c) > 0.5 then an integer x′(i) of 3 is assigned (significant impact),
followed by
(2) assessing the overall effect of the ingredient on the respective microbiome by calculating the overall impact value y with formula (II)
$$y = {\sum }_{i = 1}^{n} \frac{{x}^{′} \left(i\right)}{n} \qquad \text{formula (II)}$$
with n being the total number of microbes in the respective microbiota,
wherein a good balancing is characterized by an overall impact value y of less than 2, preferably less than 1.75, most preferably less than 1.7.

In a particularly preferred embodiment, furthermore each of x′(i) is 1 or 2.

The balancing of the growth of S. aureus in the presence of S. epidermidis is a balancing between the commensal microbe S. epidermidis over the pathogenic microbe S. aureus. In other words, the microbial growth of S. aureus is significantly more affected by the presence of 1,3-propanediol compared to the one of S. epidermidis.

Thus, specifically, said balancing is characterized in that the difference in the share of S. epidermidis, in the presence of 1,3-PDO, before and after 4 h of incubation at 37° C. is less than -25%, preferably less than -20%, most preferably less than -15%. In the absence of 1,3-propanediol, said share generally changes by more than -40%. In other words, the share of S. aureus after 4 h of incubation at 37° C. is significantly reduced by the presence of 1,3-propanediol, with respect to the share of S. aureus in the absence of 1,3-propanediol, i.e. by more than -35%, while S. epidermidis remains more or less unaffected, as outlined in the examples and illustrated in FIG. 1.

Hence, the balancing according to the present invention is characterized by maintaining the natural diversity of a co-culture of commensal microbes of dry skin or sebaceous skin, preferably while not significantly altering the absolute number of microbes and even more preferably while controlling (i.e. reducing) the growth of pathogenic microbes such as in particular S. aureus, while not negatively affecting the growth of S. epidermidis.

As a balanced microbiome leads to the maintenance of an intact skin barrier, the invention also relates to 1,3-propanediol for use in counteracting the weakening of the skin barrier, in particular of dry or sebaceous skin as defined herein, caused by a disbalance of the skin microbiome, in particular in dry or sebaceous skin, as well as in preventing or reducing the penetration of exogeneous molecules and/or microorganisms, in particular S. aureus following such a weakening.

Furthermore, as the growth of S. epidermidis and S. hominis, two particularly favourable skin microbes are increased by the presence of 1,3-propanediol compared to the respective control, the present invention also relates to the use of 1,3-propanediol as a prebiotic for S. epidermidis and/or S. hominis, preferably in the concomitant presence of C. acnes, S. capitis, S. mitis, C. simulans and M. globosa, in particular to balance the skin microbiome of sebaceous skin.

In all embodiments of the present invention, the balancing of the growth of the microbes of sebaceous skin as defined herein is preferred, as here the absolute changes in the individual shares compared to the control are within a range of ±25%, with a slight increase in the absolute number of the particularly beneficial microbes S. epidermidis and S. hominis.

Furthermore, the overall impact value is lower, as is the impact on the total number of the microbes of dry skin as defined herein.

It is important that the balancing of microbial growth allows the skin or hair to remain in a healthy state. Hence, the 1,3-propanediol or a composition comprising the 1,3-propanediol, respectively, is applied on a healthy skin or hair and maintains or improves the aesthetic aspect of hair and skin; therefore, in all embodiments of the present invention, preferably, all uses and methods are cosmetic and non-therapeutical.

Thus, in a further aspect, the present invention relates to a method of maintaining a healthy skin microbiome in dry or sebaceous skin, said method comprising the step of topically applying a cosmetic composition comprising 1,3-propanediol and optionally appreciating the effect. It is well understood that all definitions and preferences as outlined herein also apply to the method

It is particularly preferred that the uses and methods as disclosed herein are performed in the absence of a hyperbranched copolymer, such as in particular a hyperbranched of the monomers dodecenyl succinic acid anhydride, diisopropanol amine, bisdimethylaminopropyl amine having terminal groups of formula

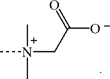

In a further embodiment, it is also particularly preferred that the uses and methods as disclosed herein are performed in the absence of one or more constituents selected from the group consisting of oligomeric and/ or polymeric fructose, monosaccharides, lactobacillus/ soy milk fermentation filtrate (and/ or extract), butanediol, 1,2-hexanediol, and pentylene glycol (1,2-pentanediol).

In all embodiments of the present invention, the 1,3-propanediol is preferably applied to the skin incorporated into a cosmetic composition.

The amount of the 1,3-propanediol in the cosmetic composition is preferably selected in the range from 0.01 to 6.0 wt.-%, preferably 0.05 to 5.0 wt.-%, more preferably 0.1 to 3.0 wt.-%, based on the total weight of the cosmetic composition. Further suitable ranges encompass 0.5 to 3 wt.-% or 1 to 2.5 wt.-%.

The term cosmetic composition’ as used in the present application refers to cosmetic compositions as defined under the heading “Kosmetika” in Römpp Lexikon Chemie, 10th edition 1997, Georg Thieme Verlag Stuttgart, New York as well as to cosmetic compositions as disclosed in A. Domsch, “Cosmetic Compositions”, Verlag für chemische Industrie (ed. H. Ziolkowsky), 4^{th} edition, 1992.

The cosmetic compositions according to the present invention are in particular compositions intended to be topically applied to mammalian keratinous tissue such as in particular to human skin or the human scalp and hair.

The cosmetic composition may be a leave-on or a rinse off cosmetic composition, and includes any product applied to a human body primarily for improving appearance, cleansing, odor control or general aesthetics.

The cosmetic compositions according to the present invention preferably further comprise a physiologically acceptable medium, that is to say a medium compatible with keratinous substances, such as the skin, mucosa, and keratinous fibres. In particular the physiologically acceptable medium is a cosmetically acceptable carrier.

The term cosmetically acceptable carrier refers to all carriers and/or excipients and/or diluents conventionally used in cosmetic compositions or pharmaceutical compositions.

The cosmetic composition may comprise further ingredients. Such ingredients are particularly surfactants, emulsifiers, thickeners, and oils. Such suitable surfactants, emulsifiers, thickeners, and oils are well known to a person skilled in the art.

Preferably, the cosmetic compositions according to the invention are in the form of a suspension or dispersion in solvents or fatty substances, or alternatively in the form of an emulsion or micro emulsion (in particular of O/W- or W/O-type), PIT-emulsion, nano emulsion, multiple emulsion (e. g. O/W/O- or W/O/W-type), pickering emulsion, hydrogel, lipogel, one- or multiphase solution or vesicular dispersion.

The cosmetic compositions in accordance with the invention can be in the form of a liquid, lotion, a thickened lotion, a gel, a cream, a milk, an ointment or a paste.

The cosmetic compositions according to the invention generally have a pH in the range from 3-10, preferably in the range from pH of 3-8, most preferred in the range from pH 3.5-7.5. The pH is adjusted by methods known to a person skilled in the art, e.g. by using an acid such as a hydroxy acid including glycolic acid, lactic acid, malic acid, citric acid and tartaric acid or a base such as e.g. sodium or potassium hydroxide or ammonium hydroxide as well as mixtures thereof.

The cosmetic compositions according to the present invention are in particular skin care preparations, functional preparations and/or hair care preparations such as most in particularly skin or hair care preparations.

Examples of skin care preparations are, in particular, light protective preparations (sun care preparations), anti-ageing preparations, preparations for the treatment of photo-ageing, body oils, body lotions, body gels, treatment creams, skin protection ointments, moisturizing preparations such as moisturizing gels or moisturizing sprays, face and/or body moisturizers, as well as skin lightening preparations.

Examples of functional preparations are cosmetic compositions containing active ingredients such as hormone preparations, vitamin preparations, vegetable extract preparations, anti-ageing preparations, and/or antimicrobial (antibacterial or antifungal) preparations without being limited thereto.

Examples of hair care preparations which are suitable according to the invention and which may be mentioned are shampoos, hair conditioners (also referred to as hair rinses), hairdressing compositions, hair tonics, hair regenerating compositions, hair lotions, water wave lotions, hair sprays, hair creams, hair gels, hair oils, hair pomades or hair brilliantines. Accordingly, these are always preparations which are applied to the hair and the scalp for a shorter or longer time depending on the actual purpose for which they are used.

In a preferred embodiment, the cosmetic compositions according to the present invention are emulsions and/or gels. Even more preferably, the cosmetic compositions are emulsions which contain an oily phase and an aqueous phase such as in particular O/W, W/O, Si/W, W/Si, O/W/O, W/O/W multiple or a pickering emulsions.

The amount of the oily phase (i.e. the phase containing all oils and fats including the polar oils) present in such emulsions is preferably at least 10 wt.-%, such as in the range from 10 to 60 wt.-%, preferably in the range from 15 to 50 wt.-%, most preferably in the range from 15 to 40 wt.-%, based on the total weight of the cosmetic composition.

The amount of the aqueous phase present in such emulsions is preferably at least 20 wt.-%, such as in the range from 20 to 90 wt.-%, preferably in the range from 30 to 80 wt.-%, most preferably in the range from 30 to 70 wt.-%, based on the total weight of the cosmetic composition.

In another preferred embodiment, the invention also provides a wipe impregnated with a composition comprising 1,3-propanediol for use on both human skin and inanimate surfaces.

Particularly important is that the 1,3-propanediol has a particular beneficial effect on the microbes of dry and sebaceous skin, which can be found on the face. the scalp and/ or the body, when combined with further skin active ingredients such as prebiotics.

Particularly preferred skin active ingredients according to the present invention are plant-derived carbohydrate compounds such as monosaccharides, disaccharides, oligosaccharides or polysaccharides, preferably fructans and galactans, as well as saccharide isomerates and polyols, particularly glycerol, sugar alcohols e.g. maltitol, sorbitol, xylitol, erythritol and isomalt, as well as vitamins such as niacinamide and mixtures thereof.

Saccharide isomerate is for example commercially available under trade name Pentavitin® from DSM Nutritional Products AG, Switzerland.

Niacinamide is for example commercially available under trade name Niacinamid PC from DSM Nutritional Products AG, Switzerland.

Examples of monosaccharide include glucose, fructose, galactose and mixtures thereof.

Examples of disaccharides include sucrose, maltose, lactose and mixtures thereof. Examples of oligosaccharides include fructo-oligosaccharides, gluco-oligosaccharide and mixtures thereof.

Fructans are a category of carbohydrate consisting of fructooligosaccharides (FOS) and inulins, while galactans consist of galactooligosaccharides (GOS). Further preferred prebiotics are resistant starch, pectin, beta-glucans, and xylooligosaccharides.

Further suitable ingredients to be combined with 1,3-propanediol in the uses and methods according to the present invention is niacinamide.

Hence, it is preferred that a composition comprising 1,3-propanediol and at least one skin active ingredient, preferably saccharide isomerate and/or niacinamide, as described above are used to balance the growth of microbes of at least two, preferably of all, microbes selected from the group consisting of M. globosa, C. tuberculostearicum, M. luteus, C. acnes, S. mitis, S. oralis, S. hominis, S. capitis, C. simulans and/or S. epidermidis, and/or to balance the microbiome of human skin and/or hair, particularly of the scalp or the scalp hair, with all the definitions and preferences as given herein.

The following examples are provided to further illustrate the compositions and effects of the present invention. These examples are illustrative only and are not intended to limit the scope of the invention in any way.

### MICROBIOME BALANCE MODEL

### 1. Assay

5 mL of TSB (Tryptic Say Broth) to which no (Ref.1) or 500 µL of 2 wt.-% of 1,3-propanediol (1,3-PDO) in PBS (Phosphate-buffered saline) have been added. Then this solutions have been inoculated by 500 µL of the respective calibrated mixture of the microbes as outlined below. Directly after inoculation and/or after 4 hours of incubation at 37° C., 100 µL have been taken and plated on an Agar gel petri dish. The microbes have then been counted based on the colony specific morphology and/or size. The results are presented below.

### 2. Calibrated Mixture of the Microbes

| | |
|---|---|
| 1. S. epidermidis/ S. aureus | CFU/ml |
| S. epidermidis | 2.3E+03 |
| S. aureus | 1.9E+03 |

| | |
|---|---|
| 2. Sebaceous skin | CFU/ml |
| P. acnes | 1.4E+05 |
| S. epidermidis | 2.4E+04 |
| S. hominis | 2.8E+04 |
| S. capitis | 1.1E+04 |
| S. mitis | 5.4E+04 |
| C. simulans | 4.3E+04 |
| M. globosa | 2.2E+04 |

| | |
|---|---|
| 3. Dry skin | CFU/ml |
| C. tuberculostearicum | 2.2E+04 |
| P. acnes | 1.4E+05 |
| S. mitis | 5.4E+04 |
| S. oralis | 5.8E+04 |
| M. luteus | 1.7E+04 |
| M. globosa | 2.2E+04 |

### Result 1: Microbiome Balancing Between S. Epidermidis and S. Aureus

Table 1 shows the counts of colony (CFU/ml) of S. epidermidis (CSE0) and S. aureus (CSA0) directly after inoculation and the counts of colony of S. epidermidis (CSE4h) and S. aureus (CSA4h) after 4 hours of incubation. All shares have been rounded.

TABLE 1

| | CSE0 | CSA0 | CSE4h | CSA4h |
|---|---|---|---|---|
| | CFU/ml (share) | | | |
| Control | 165 (89%) | 20 (11%) | 120 (40%) | 180 (60%) |
| 1,3-PDO | 235 (89%) | 30 (11%) | 170 (77%) | 50 (23%) |

In FIG. 1, the distribution of the microbes directly after inoculation (t = 0) and after 4 h (t = 4 h) are depicted. As can be retrieved from table 1, the share of S. epidermidis is significantly altered by the presence of 1,3-PDO, in favour of S. epidermidis.

### Result 2: Microbiome Balancing of Microbes of Sebaceous Skin

Table 2 shows the counts of colony (CFU/ml) of the respective microbes after 4 hours of incubation @ 37° C. All shares have been rounded.

TABLE 2

| Microbe | Control | | 1,3-PDO | | Results | |
|---|---|---|---|---|---|---|
| | CFU/ml | share | CFU/ml | share | Change | Valuation |
| C. acnes | 2836 | 24% | 2664 | 25% | +1%¹ | 1³ |
| S. epidermidis | 1800 | 16% | 2255 | 21% | +5%¹ | 2³ |
| S. hominis | 355 | 3% | 373 | 3% | ±0%¹ | 1³ |
| S. capitis | 745 | 6% | 664 | 6% | ±0%¹ | 2³ |
| S. mitis | 1591 | 14% | 1300 | 12% | -2%¹ | 2³ |
| C.simulans | 682 | 6% | 627 | 6% | ±0%¹ | 1³ |
| M. globosa | 3600 | 31% | 2900 | 27% | -4%¹ | 2³ |
| Total | 11609 | 100% | 10783 | 100% | -7%² | 1.6⁴ |

¹[value = (share control) - (share 1,3-PDO)]
²[value = (total CFU/ml 1,3-PDO) - (total CFU/ml control) / (total CFU/ml control)*100%)]
³[impact value (x(i)′]
⁴[overall impact value y]

### Result 3: Microbiome Balancing of Microbes of Dry Skin

Table 3 shows the counts of colony (CFU/ml) of the respective microbes after 4 hours of incubation @ 37° C. in the absence and in the presence of 1,3-PDO. All shares have been rounded.

TABLE 3

| Microbe | Control | | 1,3-PDO | | Results | |
|---|---|---|---|---|---|---|
| | CFU/ml | share | CFU/ml | share | Change | Valuation |
| C. tuberculostearicum | 818 | 5% | 909 | 4% | -1%¹ | 1³ |
| P. acnes | 3000 | 20% | 3200 | 15% | -4%¹ | 1³ |
| S. mitis | 3800 | 25% | 6600 | 31% | +6%¹ | 3³ |
| S. oralis | 3300 | 22% | 5700 | 27% | +5%¹ | 3³ |
| M. luteus | 782 | 5% | 873 | 4% | -1%¹ | 1³ |
| M. globosa | 3600 | 24% | 3800 | 18% | -6%¹ | 1³ |
| Total | 15300 | | 21082 | | +38%² | 1.7⁴ |

¹[value = (share control) - (share 1,3-PDO)]
²[value = (total CFU/ml 1,3-PDO) - (total CFU/ml control) / (total CFU/ml control)*100%)]
³[impact value (x(i)′]
⁴[overall impact value]

### Comparative Example

The experiments as outlined above have been repeated, using pentylene glycol (1,2-pentanediol) instead of 1,3-propanediol.

Table 4 shows the counts of colony (CFU/ml) of S. epidermidis (CSE0) and S. aureus (CSA0) directly after inoculation and the counts of colony of S. epidermidis (CSE4h) and S. aureus (CSA4h) after 4 hours of incubation with pentylene glycol. All shares have been rounded.

TABLE 4

| | CSE0 | CSA0 | CSE4h | CSA4h |
|---|---|---|---|---|
| | CFU/ml (share) | | | |
| control | 900 (77%) | 270 (23%) | 3900 (54%) | 4500 (46%) |
| pentylene glycol | 1100 (80%) | 280 (20%) | 3200 (55%( | 3900 (45%( |

As can be retrieved from table 4, the share of S. aureus is not reduced in favour of S. epidermidis by the presence of pentylene glycol.

Furthermore, the microbiome balancing of microbes of sebaceous skin was assessed in the presence of pentylene glycol.

Table 5 shows the counts of colony (CFU/ml) of the respective microbes after 4 hours of incubation @ 37° C. All shares have been rounded.

TABLE 5

| Microbe | Control | | Pentylene Glycol | | Results | |
|---|---|---|---|---|---|---|
| | CFU/ml | share | CFU/ml | share | Change | Valuation |
| C. acnes | 3900 | 39.50% | 2000 | 20% | -19.5%¹ | 3³ |
| S. epidermidis | 1500 | 15% | 3300 | 32% | +17%¹ | 3³ |
| S. hominis | 1900 | 19% | 2100 | 21% | ±2%¹ | 2³ |
| S. capitis | 880 | 9% | 1100 | 11% | ±2%¹ | 3³ |
| S. mitis | 340 | 3.50% | 380 | 4% | +0.5%¹ | 2³ |
| C.simulans | 650 | 7% | 450 | 4% | -3%¹ | 2³ |
| M. globosa | 730 | 7% | 830 | 8% | +1%¹ | 2³ |
| Total | 9900 | 100% | 10160 | 100% | +2.60%² | 2.4⁴ |

¹[value = (share control) - (share Pentylene Glycol)]
²[value = (total CFU/ml Pentylene Glycol) - (total CFU/ml control) / (total CFU/ml control)*100%)]
³[impact value (x(i)′]
⁴[overall impact value y]

As can be retrieved from table 5 above, in contrast to 1,3-propanediol, pentylene glycol does alter the share of the microbes of sebaceous skin to a greater extend and can thus not be considered as being particularly microbiome friendly.

### FORMULATION EXAMPLE

Wipe impregnated 1

| INCI name | % w / w | % w/w range |
|---|---|---|
| Aqua | Ad 100 | Add 100 |
| Disodium EDTA | 0.20 | 0.1-0.5 |
| Aqua and Saccharide isomerate and citric acid and sodium citrate | 1.00 | 0.1-3.00 |
| 1,3 Propanediol - TILAMAR PDO | 2.00 | 0.5-5.00 |
| PEG-6 Caprylic/Capric glycerides | 2.00 | 1.00-3.00 |
| Microcrystalline Cellulose and Cellulose gum | 0.50 | 0.1-1.00 |
| Citrci acid | 0.20 | 0.05-0.50 |
| Aqua and sodium benzoate and potassium sorbate | 1.50 | 0.30-2.00 |
| Tocpherol | 0.10 | 0.05-1.00 |

Wipe impregnated 2

| INCI name | % w / w | % w/w range |
|---|---|---|
| Aqua | Add 100 | Add 100 |
| 1,3 Propanediol - TILAMAR PDO | 3.00 | 0.5-5.00 |
| Sodium gluconate | 0.10 | 0.05-0.50 |
| Aqua and and Glycerin and Levulinic acid and sodium levulinate | 1.00 | 0.50.5.00 |
| Sodium benzoate | 0.25 | 0.05-0.50 |
| Aqua and Glycerin and Epilobium fleischeri flower/leaf/stem extract and citric acid | 0.50 | 0.1-1.50 |
| Caprylyl/Capryl Glucoside | 1.50 | 0.80-2.00 |
| Aqua and Sodium hydroxide (10% solution) | 0.50 | 0.20-0.80 |

1. A cosmetic, non-therapeutic use of a cosmetic composition comprising 1,3-propanediol in an amount selected in the range from 0.01 to 6.0 wt.-%, based on the total weight of the cosmetic composition for balancing the growth of at least two microbes of dry or sebaceous skin by not unproportionally affecting the growth of anyone thereof and the growth of S. aureus in the presence of S. epidermidis by inhibiting the growth of S. aureus to a higher extent than the one of S. epidermidis.

2. The use according to claim 1, wherein the microbes of dry skin are selected from C. tuberculostearicum, C. acnes, S. mitis, S. oralis, M. luteus and M. globosa and the microbes of sebaceous skin are selected from the group of C. acnes, S. epidermidis, S. hominis, S. capitis, S. mitis, C. simulans and M. globosa.

3. The use according to claim 1, wherein the balancing of the growth of the at least two, preferably all microbes of dry or sebaceous skin is characterised in that the difference in the share of each individual microbe, compared to the respective share in the absence of 1,3-propanediol, after 4 h of incubation at 37° C., is in the range of ±15%, preferably in the range of ±10%, most preferably in the range of ±7.5%.

4. The use according to claim 1, wherein the balancing of the growth of the at least two, preferably all microbes of dry or sebaceous skin is characterised in that the difference in the total number of said at least two, preferably all microbes, compared to the respective total number in the absence of 1,3-propanediol, after 4 h of incubation at 37° C., is within a range of ±50%, preferably ±40%, more preferably ±15%, most preferably ±10%.

5. The use according to claim 2, wherein the difference in total number of each of the at least two, preferably all microbes of dry skin are within the range of
±20%, preferably 0% to 20%, more preferably +5% to +15%, most preferably +7.5% to +15% for C. tuberculostearicum,
±15%, preferably 0% to +15%, more preferably +2.5% to +10%, most preferably +5% to +10% for C. acnes,
±75%, preferably 0% to +75%, more preferably +25% to +75%, most preferably +50% to +75% for S. mitis and S. oralis,
±20%, preferably 0% to 20%, more preferably +5% to +15%, most preferably +7.5% to +15% for M. luteus, and/or
±15%, preferably 0% to +15%, more preferably +2.5% to +10%, most preferably +5% to +10% for M. globosa
with respect to the total number of each of the microbes in the absence of 1,3-propanediol.

6. The use according to claim 2, wherein the difference in total number of each of the at least two, preferably all microbes of sebaceous skin, is within the range of
±15%, preferably +5% to -15%, more preferably 0% to -10%, most preferably -2.5% to -7.5% for C. acnes,
±35%, preferably +5% to +35%, more preferably +15% to + 30%, most preferably +20% to +30% for S. epidermidis,
±15%, preferably 0% to +15%, more preferably +2.5% to + 10%, most preferably +2.5% to +7.5% for S. hominis,
±20%, preferably 0% to -20%, more preferably -7.5% to -15%, most preferably -10% to -15% for S. capitis,
±25%, preferably -5% to -25%, more preferably -10% to -23%, most preferably -15% to -20% for S. mitis,
±15%, preferably 0% to -15%, more preferably -2.5% to -10%, most preferably -5% to -10% for C. simulans, and/or
±25%, preferably -5% to -25%, more preferably -10% to -23%, most preferably -15% to -23% for M. globosa
with respect to the total number of each of the microbes in the absence of 1,3-propanediol.

7. The use according to claim 1, wherein the balancing of the growth of S. aureus in the presence of S. epidermidis is characterized in that the difference in the share of S. epidermidis, in the presence of 1,3-propanediol, before and after 4 h of incubation at 37° C., is less than -25%, preferably less than -20%, most preferably less than -15%.

8. The use according to claim 1, wherein a healthy skin microbiome is maintained.

9. A method of maintaining a healthy skin microbiome in dry or sebaceous skin, said method comprising the step of topically applying a cosmetic composition comprising 1,3-propanediol in an amount selected in the range from 0.01 to 6.0 wt.-%, based on the total weight of the cosmetic composition.

10. A method according to claim 9, wherein the healthy microbiome is maintained by balancing at least two microbes selected from the group consisting of M. globosa, C. tuberculostearicum, M. luteus, C. acnes, S. mitis, S. oralis, S. hominis, S. capitis, C. simulans and/or S. epidermidis.

11. A method according to claim 9, wherein the healthy microbiome is maintained
a. in dry skin by balancing the growth of C. tuberculostearicum, C. acnes, S. mitis, S. oralis, M. luteus and M. globosa,
b. in sebaceous skin by balancing the growth of C. acnes, S. epidermidis, S. hominis, S. capitis, S. mitis, C. simulans and M. globosa.

12. 1,3-Propanediol for use in counteracting the weakening of the skin barrier caused by a disbalance of the skin microbiome as well as in preventing or reducing the penetration of exogeneous molecules and/or microorganisms following such a weakening.

13. The use according to claim 12, wherein the exogeneous molecules are irritant, toxic or allergenic substances.

14. The use according to claim 12, wherein the microorganisms are pathogenic microbes, in particular S. aureus.

15. Use of 1,3-propanediol as a prebiotic for S. epidermidis and/or S. hominis, preferably in the concomitant presence of C. acnes, S. capitis, S. mitis, C. simulans and M. globosa and/or S. aureus.
